(19) United States
(12) Patent Application Publication
(10) Pub. No.: US 2005/0060816 A1
(43) Pub. Date: Mar. 24, 2005

(54) Composition comprising at least one conductive polymer and at least one oxidation dye, and process for the use thereof

(76) Inventors: Isabelle Rollat-Corvol, Paris (FR); Henri Samain, Bievres (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/892,254

(22) Filed: Jul. 16, 2004

Related U.S. Application Data
(60) Provisional application No. 60/492,297, filed on Aug. 5, 2003.

(30) Foreign Application Priority Data
Jul. 16, 2003 (FR) 03 08675

(51) Int. Cl. A61K 7/13

(52) U.S. Cl. 8/405

(57) ABSTRACT

The disclosure relates to compositions comprising, in a cosmetically acceptable medium, (a) at least one oxidation dye and (b) at least one conductive polymer. The disclosure also relates to ready-to-use compositions comprising the abovementioned compositions, processes using such a ready-to-use composition, and methods of using the compositions for giving keratin fibers an optical effect.

This application claims the benefit of U.S. Provisional Application No. 60/492,297, filed Aug. 5, 2003.

The present disclosure provides compositions comprising, in a cosmetically acceptable medium, at least one conductive polymer and at least one oxidation dye. The present disclosure also provides processes for treating keratin fibers using the abovementioned compositions. In addition, the present disclosure provides methods of using such compositions to impart to keratin fibers an optical effect.

The present disclosure relates to the field of hair dyeing, including permanent dyeing using oxidation dyes.

Oxidation dyes typically comprise at least one oxidation dye precursor (also known as an oxidation base), optionally combined with at least one coupler that may allow the shade obtained with the precursor or precursors to be varied.

Generally, oxidation dye precursors are chosen from ortho- or para-phenylenediamines, ortho- or para-aminophenols, and heterocyclic compounds such as diaminopyrazole derivatives. They are colorless or weakly colored compounds which, when combined with oxidizing products, lead to colored compounds via a process of oxidative condensation.

Typically, the couplers are chosen from aromatic meta-diamines, meta-aminophenols, meta-diphenols and certain heterocyclic compounds such as indole compounds, etc.

It is known practice to incorporate additional agents into dyeing compositions in an attempt to improve the optical effects given to keratin fibers after the dyeing process, for example sheen effects.

For example, hydrophobic lubricating substances, such as organic oils, waxes, and silicones, are often used to give the fibers sheen. However, the sheen effect obtained may lack intensity and generally give the fibers an artificial appearance.

In addition, such compositions may have the drawback of giving the fibers a greasy and/or tacky feel. The presence of these types of compounds may also limit the uptake of the dye into the fibers and consequently result in less intense colorations.

The aim of the present disclosure is thus to provide compositions comprising at least one oxidation dye, which give treated keratin fibers a particular optical effect without at least one of the drawbacks encountered with the standard compositions, and at the same time maintain good coloration properties, such as fast, strong and sparingly selective colors. The fibers may also advantageously have a soft, pleasant feel.

In one embodiment, disclosed herein are compositions comprising, in a cosmetically acceptable medium:
(a) at least one oxidation dye, and
(b) at least one conductive polymer.

In a second embodiment, the present disclosure relates to ready-to-use compositions comprising the composition described above and at least one oxidizing agent.

Another embodiment relates to, processes for treating keratin fibers, such as human keratin fibers, e.g., hair, comprising:
a) applying the ready-to-use composition described above to wet or dry fibers and leaving it on the fibers for a time sufficient to develop a coloration,
b) optionally rinsing the fibers,
c) washing and rinsing the fibers, and
d) drying the fibers or leaving the fibers to dry.

In still another embodiment, the use of a composition comprising at least one conductive polymer and at least one oxidation dye to impart to keratin fibers an optical effect is provided

The compositions according to the disclosure may uniformly give fibers a particular optical effect, for example, a sheen that may be more intense, more natural and more aesthetic than with the means of the prior art.

Moreover, when the conductive polymers present in the composition absorb in the visible spectrum, optical effects, for example sheen, and color are obtained simultaneously.

In certain embodiments, it is also possible to broaden the range of colors that may be obtained. In certain other embodiments, the contents of oxidation dyes may be optimized.

However, other characteristics and advantages of the disclosure will be apparent from reading the description and the examples that follow.

Unless otherwise indicated, the limits of a range of values should be understood to form part of the range.

As used herein, the term “optical effect” includes sheen, color, metallic, goniochromatic, and moiré effects.

Sheen, or specular reflection, corresponds to the light intensity reflected at an angle alpha (α) when the lock of hair is illuminated under an angle negative alpha (−α). A twenty degree (20°) angle alpha (α) is conventionally used to measure the specular reflection, or sheen. The sheen may be measured using a glossmeter as described, for example, in ISO standard 2813-1994 from AFNOR (August 1994, amended February 1997).

Conductive Polymers

As used herein, the term “conductive polymer” means a molecular structure in which the monomer or monomers has high electron delocalization and whose arrangement in the polymer skeleton allows the pi (90) orbitals to overlap. This chemical characteristic is characterized by electrical conduction, which may or may not be accompanied by absorption in the UV-visible or infrared spectrum.

As used herein, the expression “conductive polymer absorbing in the visible spectrum” means any conductive polymer having a non-zero absorbance in the wavelength ranging from 400 to 800 nm, even if the absorption maximum or maxima of the polymer are outside this range.

The conductive polymers used in the compositions, processes, and methods described herein are conductive polymers that are soluble or dispersible in a cosmetic medium suitable for use.

A polymer is said to be soluble in a medium if it forms an isotropic clear liquid in the medium comprising water or a water/solvent mixture at 25° C., throughout all or part of a concentration range from 0.01% to 50% by weight of conductive polymer.

For example, the conductive polymers may be soluble or dispersible in an aqueous medium, such as water.

A polymer is said to be dispersible in a medium comprising water or a water/solvent mixture if, at 0.01% by weight at 25° C., it forms a stable suspension of fine, generally spherical particles. The mean size of the particles constituting the dispersion is less than about 1 micrometer (μm), more generally ranging from 5 to 400 nanometers (nm), for example, from 10 to 250 nm. The particle sizes may be measured by light scattering.

In one embodiment, these polymers do not require the use of a dispersant.

The conductive polymers may be in a form that is soluble in the medium of the composition.

Furthermore, the polymers may have a conductivity ranging from 10⁻⁵ to 5×10⁵ siemens/cm, for example, from 10⁻³ to 10⁵ siemens/cm or from 10⁻¹ to 10⁴ siemens/cm.

The conductivity may be measured using a current generator (RM2 Test Unit sold by Jandel) equipped with a four-point measuring head (Universal four-point probes sold by Jandel). The four points, aligned and separated by the same space (d), are applied by simple pressure to the sample to be analyzed. A current (I) is injected via the outer points using the current source, thus creating a variation in potential. The voltage U is measured between the two inner points connected to the voltmeter of the current generator.

In this configuration, the conductivity of the sample (sigma, σ) in siemens/cm is given by the following expression:
σ=(K×I)/(U×e)
in which:
K is a coefficient depending on the position of the contacts on the surface of the sample. When the points are aligned and equidistant, K is equal to: π/log(2);
I is the value of the injected current in amperes;
U is the measured voltage value in volts; and
e is thickness of the sample in cm.

This expression can be used only when the thickness of the material is small compared to the distance (d) existing between two points, for example, when (e/d)<0.25. In order to obtain sufficiently small thicknesses to be able to calculate the conductivity of the material, the measurements may be performed on a non-conductive support (for example a glass slide) coated with the material to be analyzed, obtained by, for example, evaporation of a dilute solution. In order to improve the homogeneity of the coating to be analyzed, one may use the deposition technique known as spin coating.

According to one embodiment, the conductive polymers present in the composition are chosen from polymers comprising at least one repeating unit of the following formulas:
anilines of formula (I): 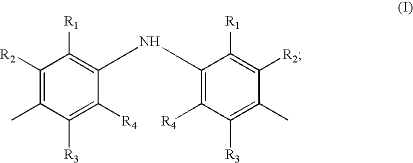
pyrroles of formulas (IIa) and (IIb): 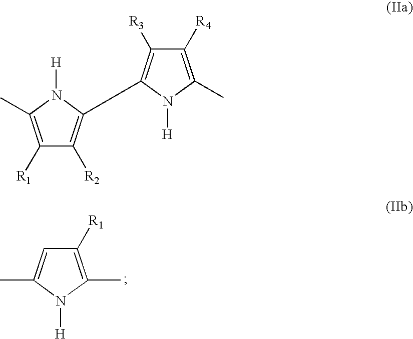
thiophenes and bisthiophenes of formulas (IIIa), (IIIb) and (IIIc): 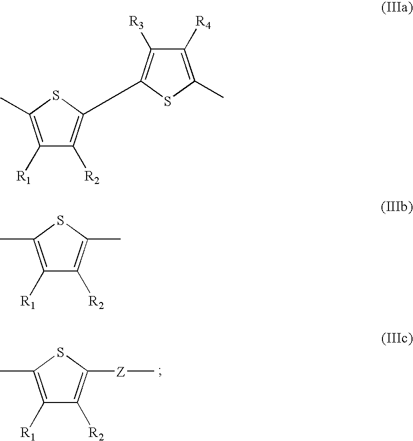
furans of formula (IV) 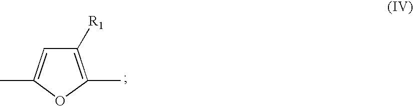
para-phenylene sulfides of formula (V): 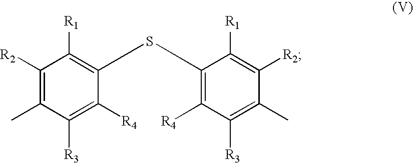
para-phenylenevinylens of formula (VI): 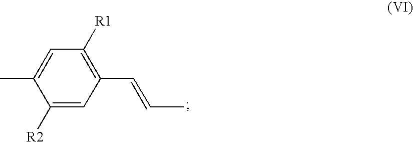
   indoles of formula (VII): 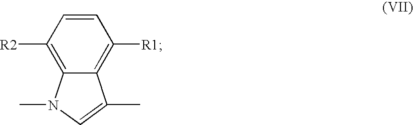
aromatic amides of formulas (VIIIa), (VIIIb), (VIIIc) and (VIIId): 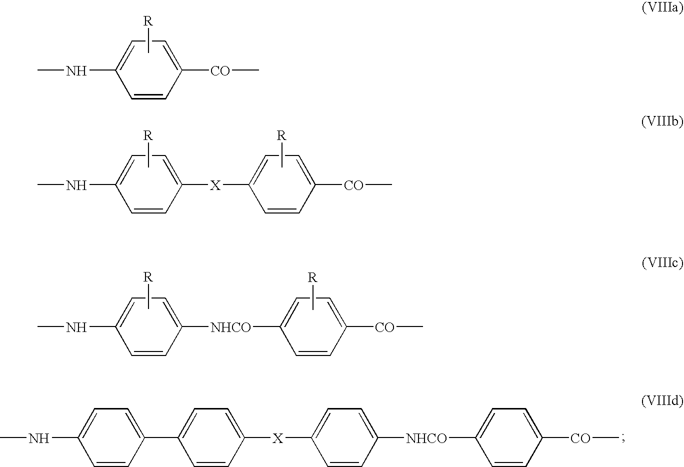
aromatic hydrazides of formulas (IXa), (IXb) and (IXc): 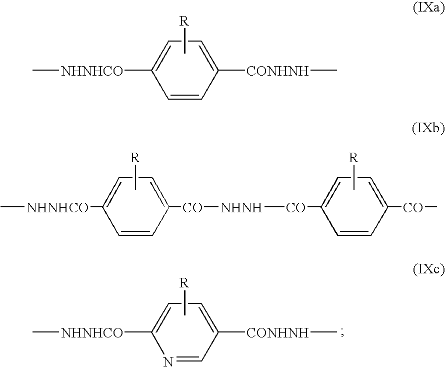
aromatic azomethines of formulas (Xa), (Xb) and (Xc): 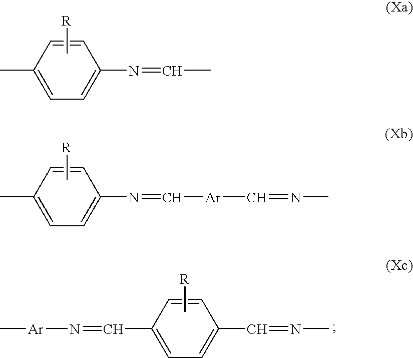
and
aromatic esters of formulas (XIa), (XIb) and (XIc): 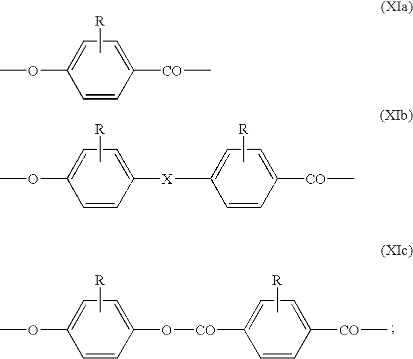
in which formulas (I) to (XI):
the radicals R, R₁, R₂, R₃, and R₄, which may be identical or different, are each chosen from hydrogen and the radicals —R′, —OR′, —COOR′, and —OCOR′, wherein R′ is chosen from linear and branched C₁-C₂₀ alkyl radicals, halogen atoms, nitro radicals, cyano radicals, cyanoalkyl radicals, and solubilizing groups optionally comprising a spacer group that bonds to the ring;
Ar is chosen from a radical comprising a monoaromatic or a polyaromatic radical; X is chosen from —O— and —S— atoms and —NHCO—, —SO₂—, —N═N—, —C(CH₃)₂—, —CH₂—, —CH═CH—, and —CH═N— radicals; and Z is chosen from —CH═CH— and —C≡C— radicals.

Ar may be chosen, for example, from at least one radical chosen from the following:

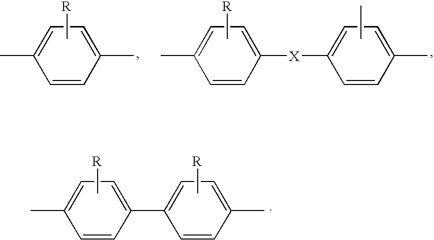

As used herein, the term “solubilizing group” means a group that ensures the dissolution of the molecule in the cosmetic medium, such that the polymer has a conductive nature after drying the composition.

The conductive polymers present in the compositions disclosed herein may comprise at least one repeating unit comprising at least one solubilizing group, and at least one other such units without solubilizing groups.

Examples of solubilizing groups include, but are not limited to:
carboxylic acids (—COOH),
carboxylate radicals (—COO^{-M}⁺) wherein M is chosen from alkali metals, such as sodium and potassium; alkaline-earth metals; organic amines, such as primary, secondary and tertiary amines; alkanolamines; and amino acids,
sulfonic radicals (—SO₃H),
sulfonate radicals (—SO₃⁻M⁺) wherein M is as described above,
primary, secondary and tertiary amine radicals,
quaternary ammonium radicals such as —N(R′)₃⁺Z wherein Z is chosen from Br and Cl atoms, and from (C₁-C₄)alkyl-OSO₃ radicals wherein each R′, which may be identical or different, is chosen from linear and branched C₁ to C₂₀ alkyls, or two may form a heterocycle together with the nitrogen,
hydroxyl radicals, and
poly((C₂-C₃)alkylene oxide) radicals.

The carboxylic or sulfonic acid functions may be optionally neutralized with a base, such as sodium hydroxide, 2-amino-2-methylpropanol, triethylamine or tributylamine.

The amine radicals may be optionally neutralized with a mineral acid, such as hydrochloric acid, or with an organic acid, such as acetic acid or lactic acid.

In addition, the solubilizing radicals may optionally be connected to the ring via a spacer group, such as the radicals —R″—, —OR″—, —OCOR″—, and —COOR″— wherein R′ is chosen from linear and branched C₁-C₂₀ alkyl radicals optionally comprising at least one heteroatom, for example oxygen.

In some embodiments, the radicals R, R₁, R₂, R₃, and R₄, which may be identical or different, are chosen from hydrogen and R′, —OR′, —OCOR′ and —COOR′ radicals wherein R′ is chosen from linear and branched C₁-C₆ alkyl radicals, and from the following neutralized or non-neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, —[(CH₂)₂O]ₓCH₂CH₂OCH₃ with x being an average number between 0 and 200.

The number (n) of repeating units in the polymer may range from 5 to 10,000, from 5 to 1000, from 10 to 1000 or even from 20 to 700.

In some embodiments, at least one of R, R₁, R₂, R₃, and R₄ of the conductive polymer is a solubilizing group.

In one particular embodiment, the conductive polymer used comprises at least one solubilizing group per repeating unit. Thus, in this embodiment, at least one radical from among R, R₁, R₂, R₃, and R₄ is a solubilizing group.

In certain embodiments, the conductive polymer is soluble in the medium of the composition.

Conductive polymers that may be present in the compositions disclosed herein are well known to those skilled in the art and are described, for example, in Handbook of Organic Conductive Molecules and Polymers; Wiley: New York, 1997; Vol 1, 2, and 3 and Can. J. Chem. Vol 64,1986.

Polythiophenes and syntheses thereof are described in Rasmussen S. C., Pickens J. C. and Hutchison J. E., “A new, general approach to tuning the properties of functionalized polythiophenes: The oxidative polymerization of monosubstituted bithiophenes” Chem. Mater. 1998,10 (7), 1990-1999 and Rasmussen S. C., Pickens J. C. and Hutchison J. E., “Highly conjugated, water-soluble polymers via direct oxidative polymerization of monosubstituted bithiophenes” Macromolecules 1998, 31, 933-936. In addition to polymerization via chemical or electrochemical oxidation, they may also be obtained by polycondensation (dihalothiophene; catalysis with nickel or palladium complexes); via Suzuki coupling (coupling between a halogen function, for example bromine, and a boronic acid; catalysis (palladium complex and base) resulting in AA-BB type coupling (reaction of monomers of the type A-X-A with B-X′-B) or A-B type coupling (reaction of several monomers of the type A-X-B); via Stille coupling (formation of a carbon-carbon bond in the presence of a Pd-based catalyst: AA-BB or A-B type coupling); via Reike polymerization (organozinc in the presence of a nickel complex); via polymerization of McCulloch type; etc.

Other conductive polymers that may be present in the compositions disclosed herein are described in international patent application publication WO 99/47570.

Conductive polymers that are suitable for use in the compositions, processes, and uses described herein include the polymers corresponding to formulas (IIIa), (IIIb) and (IIIc) in which the solubilizing groups are, for example, chosen from carboxylic acid groups; sulfonic acid groups; tertiary amine radicals; quaternary ammonium radicals such as —N(R′)₃⁺Z⁻ wherein Z is chosen from Br and Cl atoms and (C₁-C₄)alkyl-OSO₃ radicals and each R′, which may be identical or different, is chosen from linear and branched C₁ to C₂₀ alkyl radicals, or two of them form a heterocycle together with the nitrogen. The groups are optionally connected to the ring via a spacer group. The carboxylic or sulfonic acid functions may optionally be neutralized.

The polymerization may be performed via chemical or electrochemical oxidation of the corresponding thiophene monomers or via polycondensation.

The polythiophenes of formulas (IIIa) and (IIIb) may be obtained, for example, by polymerization via oxidation (for example with FeCl₃ catalysis); via polycondensation of dihalothiophene catalyzed with nickel or palladium complexes (e.g., NiCl₂(dppe)₂); via Suzuki coupling (coupling between a halogen function, for example bromine, and a boronic acid), catalysis: palladium complex and base; this then gives AA-BB type coupling (reaction of monomers of the type A-X-A with B-X′-B) or A-B type coupling (reaction of several monomers of the A-X-B type); via Stille coupling (formation of a carbon-carbon bond formed in the presence of a Pd-based catalyst: AA-BB or A-B type); via Reike polymerization (organozinc in the presence of a nickel complex); via McCulloch type polymerization, etc.

The vinylene polythiophenes of formula (IIIc) wherein Z is —CH≡CH— may be obtained via Gilch polymerization in the presence of a strong base (potassium tert-butoxide) of 2,5-bis(bromoalkylene)thiophene; via polymerization by the Wessling method via the use of a precursor based on sulfonium salts and pyrolysis; and via a Wittig-Horner Wittig reaction.

The ethynylene polythiophenes of formula (IIIc) wherein Z is —C≡C— may be obtained by Heck-Sonogashira coupling (A-BB or A-B type); formation of a carbon-carbon bond between a terminal acetylenic (or true acetylenic) function and a bromo or iodo function, catalyzed with a palladium/copper complex (PdCl₂(PPh₃)₃, Cul or Cu(OAc)₂) in the presence of a base such as triethylamine, diisopropyl amine, piperidine, etc.; or via metathesis of alkynes in the presence of a molybdenum complex (Mo(CO)₆).

In general, the functionalization of the polythiophenes (introduction of the solubilizing or non-solubilizing group(s)), is performed on the monomers before they are polymerized.

In certain cases, the solubilizing group is obtained after working up the polymer. For example, carboxylic acid functions may be obtained by hydrolysis of the corresponding esters.

The solubilizing groups may be chosen from carboxylic acid groups; sulfonic acid groups; tertiary amine radicals; quaternary ammonium radicals such as —N(R′)₃⁺Z wherein Z is chosen from Br and Cl atoms and (C₁-C₄)alkyl-OSO₃ radicals and each R′, which may be identical or different, is chosen from linear and branched C₁-C₂₀ alkyl radicals, optionally connected to the ring via a spacer group, e.g., a C₁-C₂₀ alkyl radical; and salts thereof. The carboxylic or sulfonic acid functions may optionally be neutralized.

According to one embodiment, the conductive polymer corresponds to formula (IIIa), (IIIb) or (IIIc), in which at least one of R₁, R₂, R₃, and R₄ of formula (IIIa) or R₁ or R₂ of formulas (IIIb) or (IIIc) is chosen from carboxylic acid solubilizing groups, in a neutralized or non-neutralized form, optionally connected to the ring via a spacer group, such as a linear or branched C₁-C₂₀ alkyl radical, and wherein the other radical or radicals are hydrogen atoms.

The conductive polymers are generally present in the composition in proportions of at least 0.001% by weight, at least 0.01% by weight, at least 0.1% by weight, or even least 0.5% by weight, relative to the total weight of the composition. Moreover, the content of conductive polymer may be up to 50% by weight, up to 30% by weight, up to 20% by weight, and even up to 10% by weight, relative to the total weight of the composition.

In one embodiment, the content of conductive polymer ranges from 0.1% to 50% by weight, from 0.1% to 30% by weight, or even from 0.5% to 10% by weight, relative to the total weight of the composition.

Oxidation Dyes

The oxidation dyes that may be used are chosen from oxidation bases, couplers, or both oxidation bases and couplers.

In certain embodiments, the compositions comprise at least one oxidation base.

The oxidation bases are chosen from those conventionally known in oxidation dyeing, for example, ortho-phenylenediamines and para-phenylenediamines, double bases, ortho-aminophenols and para-aminophenols, heterocyclic bases such as those described below, and acid addition salts thereof.

(I) The oxidation bases may be chosen from the para-phenylenediamines of formula (A) below, and acid addition salts thereof: 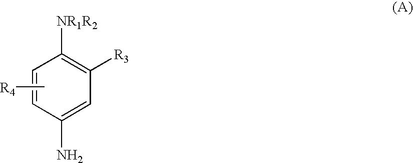
wherein:
R₁ is chosen from a hydrogen atom, C₁-C₄ alkyl radicals, C₁-C₄ monohydroxyalkyl radicals, C₂-C₄ polyhydroxyalkyl radicals, (C₁-C₄)alkoxy(C₁-C₄)alkyl radicals, and C₁-C₄ alkyl radicals substituted with a nitrogenous, phenyl or 4′-aminophenyl group;
R₂ is chosen from a hydrogen atom, C₁-C₄ alkyl radicals, C₁-C₄ monohydroxyalkyl radicals, C₂-C₄ polyhydroxyalkyl radicals, (C₁-C₄)alkoxy(C₁-C₄)alkyl radicals, and C₁-C₄ alkyl radicals substituted with a nitrogenous group; or
R₁ and R₂ may form, together with the nitrogen atom to which they are attached, a 5- or 6-membered nitrogenous heterocycle optionally substituted with at least one group chosen from alkyl, hydroxyl and ureido groups;
R₃ is chosen from a hydrogen atom, halogen atoms, such as a chlorine atom, C₁-C₄ alkyl radicals, sulfo radicals, carboxy radicals, C₁-C₄ monohydroxyalkyl radicals, C₁-C₄ hydroxyalkoxy radicals, acetylamino(C₁-C₄)alkoxy radicals, mesylamino(C₁-C₄)alkoxy radicals, and carbamoylamino(C₁-C₄)alkoxy radicals; and
R₄ is chosen from a hydrogen atom, halogen atoms, and C₁-C₄ alkyl radicals.

The nitrogenous groups of formula (A) above include, for example, amino, mono(C₁-C₄)alkylamino, di(C₁-C₄)alkylamino, tri(C₁-C₄)alkylamino, monohydroxy(C₁-C₄)alkylamino, imidazolinium, and ammonium radicals.

The para-phenylenediamines of formula (A) above, include, for example, para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 2-methyl-1-N-β-hydroxyethyl-para-phenylenediamine, and acid addition salts thereof.

Among the para-phenylenediamines of formula (A) may be mentioned, for example, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, and acid addition salts thereof.

As used herein, the term “double bases” means compounds comprising at least two aromatic nuclei bearing amino and/or hydroxyl groups.

(II) The double bases that may be used as oxidation bases in the dye compositions described herein include, for example, compounds corresponding to formula (B) below, and acid addition salts thereof: 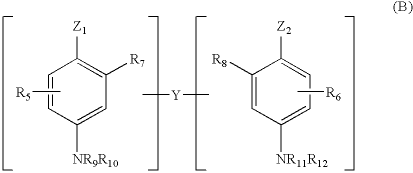
wherein:
Z₁ and Z₂, which may be identical or different, are each chosen from hydroxyl and —NH₂ radicals which may be substituted with a C₁-C₄ alkyl radical or with a linker arm Y; the linker arm Y is a linear or branched alkylene chain containing from 1 to 14 carbon atoms, which may be interrupted by or terminated with at least one entity chosen from nitrogenous groups and heteroatoms such as oxygen, sulfur or nitrogen atoms, and optionally substituted with at least one radical chosen from hydroxyl and C₁-C₆ alkoxy radicals;
R₅ and R₆, which may be identical or different, are chosen from a hydrogen atom, halogen atoms, C₁-C₄ alkyl radicals, C₁-C₄ monohydroxyalkyl radicals, C₂-C₄ polyhydroxyalkyl radicals, C₁-C₄ aminoalkyl radicals, and a linker arm Y; and
R₇, R₈, R₉, R₁ ₀, R₁₁ and R₁₂, which may be identical or different, are chosen from a hydrogen atom, a linker arm Y, and a C₁-C₄ alkyl radical,
wherein the compounds of formula (B) comprise only one linker arm Y per molecule.

The nitrogenous groups of formula (B) include, for example, amino, mono(C₁-C₄)alkylamino, di(C₁-C₄)alkylamino, tri(C₁-C₄)alkylamino, monohydroxy(C₁-C₄) alkylamino, imidazolinium, and ammonium radicals.

The double bases of formula (B) above, include, for example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)-tetramethylenediamine, N,N′-bis(β-hydroxyethyl)-N,N′-bis(4-aminophenyl)-tetramethylened iamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,5-dioxaoctane, and acid addition salts thereof.

In certain embodiments, the double bases of formula (B), are chosen from N,N′-bis(β-hydroxyethyl)-N,N′-bis(4′-aminophenyl)-1,3-diaminopropanol, 1,8-bis(2,5-diaminophenoxy)-3,5-dioxaoctane, and acid addition salts thereof.

(III) The oxidation bases that may be used include para-aminophenols corresponding to formula (C) below, and acid addition salts thereof: 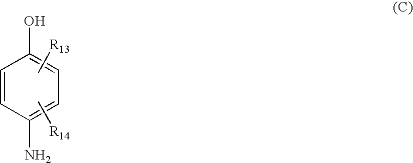
wherein:
R₁₃ is chosen from a hydrogen atom, halogen atom s such as fluorine, and C₁-C₄ alkyl, C₁-C₄ monohydroxyalkyl, (C₁-C₄)alkoxy(C₁-C₄)alkyl, C₁-C₄ aminoalkyl and hydroxy(C₁-C₄)alkylamino(C₁-C₄)alkyl radicals; and
R₁₄ is chosen from a hydrogen atom, halogen atoms such as fluorine, and C₁-C₄ alkyl, C₁-C₄ monohydroxyalkyl, C₂-C₄ polyhydroxyalkyl, C₁-C₄ aminoalkyl, C₁-C₄ cyanoalkyl and (C₁-C₄)alkoxy(C₁-C₄)alkyl radicals.

The para-aminophenols of formula (C) include, for example, para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, and acid addition salts thereof.

(IV) The ortho-aminophenols that may be used as oxidation bases in the context of the present disclosure include, for example, 2-aminophenol, 2-amino-1-hydroxy-5-methylbenzene, 2-amino-1-hydroxy-6-methylbenzene, 5-acetamido-2-aminophenol, and acid addition salts thereof.

(V) The heterocyclic bases that may be used as oxidation bases in the dye compositions include, but are not limited to, pyridine derivatives, pyrimidine derivatives, pyrazole derivatives, and acid addition salts thereof.

Pyridine derivatives include the compounds described, for example, in patents GB 1,026,978 and GB 1,153,196 as well as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and acid addition salts thereof.

Pyrimidine derivatives include the compounds described, for example, in patents DE 2,359,399, JP 88-169571, JP 91-10659, and published patent application WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine, and 2,5,6-triaminopyrimidine, as well as pyrazolopyrimidine derivatives such as those disclosed in patent application FR 2,750,048 including pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5-a]pyrimidin-3-ylamino)ethanol; 2-[(3-aminopyrazolo[1,5-a]pyrimidin-7-yl)(2-hydroxyethyl)amino]ethanol; 2-[(7-aminopyrazolo[1,5-a]pyrimid in-3-yl)(2-hydroxyethyl)amino]ethanol; 5,6-dimethylpyrazolo[1,5-a]pyrimidine-3,7-diamine; 2,6-dimethylpyrazolo[1,5-a]pyrimidine-3,7-diamine; 2,5,N7,N7-tetramethylpyrazolo[1,5-a]pyrimid ine-3,7-diamine; 3-amino-5-methyl-7-imidazolylpropylaminopyrazolo[1,5-a]pyrimidine; and acid addition salts thereof, as well as tautomeric forms thereof when a tautomeric equilibrium exists, and addition salts thereof.

Pyrazole derivatives include, for example, the compounds described in patents DE 3,843,892, DE 4,133,957 and published patent applications WO 94/08969, WO 94/08970, FR 2,733,749, and DE 1,9543,988, such as 4,5-diamino-1-methyl-pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-(β-hydroxyethyl)pyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and acid addition salts thereof.

The oxidation base(s) may be present in an amount ranging from 0.0005% to 12% by weight relative to the weight of the composition, such as from 0.005% to 8% by weight relative to the weight of the composition.

The couplers that may be used in the composition are those conventionally used in oxidation dye compositions, e.g., meta-aminophenols, meta-phenylenediamines, meta-diphenols, naphthols and heterocyclic couplers such as, for example, indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines, and acid addition salts thereof.

The couplers may be chosen from, for example, 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2-amino-3-hydroxypyrid ine, 3,6-dimethylpyrazolo[3,2-c]-1,2,4-triazole, 2,6-dimethylpyrazolo[1,5-b]-1,2,4-triazole, and acid addition salts thereof.

When present, the couplers are present in an amount ranging from 0.0001% to 10% by weight relative to the total weight of the composition, for example, from 0.005% to 5% by weight.

In general, the acid addition salts of the oxidation bases and couplers may be chosen from hydrochloride, hydrobromide, sulfate, tartrate, lactate, and acetate salts.

Surfactants

The cosmetic compositions disclosed herein may also comprise at least one surfactant, such as anionic, amphoteric, nonionic, zwifterionic and cationic surfactants, alone or as mixtures.

Examples of suitable surfactants include the following:
(i) Anionic surfactant(s):

Examples of anionic surfactants that may be used, alone or as mixtures, include, but are not limited, to salts (such as alkali metal salts, e.g., sodium salts, ammonium salts, amine salts, amino alcohol salts, and magnesium salts) of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; (C₆-C₂₄)alkyl sulfosuccinates, (C₆-C₂₄)alkyl ether sulfosuccinates, (C₆-C₂₄)alkylamide sulfosuccinates; (C₆-C₂₄)alkyl sulfoacetates; (C₆-C₂₄)acyl sarcosinates; and (C₆-C₂₄)acyl glutamates. It is also possible to use (C₆-C₂₄)alkylpolyglycoside carboxylic esters such as alkylglucoside citrates, alkylpolyglycoside tartrate and alkylpolyglycoside sulfosuccinates, alkylsulfosuccinamates; acyl isethionates and N-acyl taurates, alkyl or acyl radicals of these compounds containing, for example, from 12 to 20 carbon atoms. The aryl radical may be, for example, a phenyl or benzyl group. The anionic surfactants which may be used include fatty acid salts such as oleic, ricinoleic, palmitic and stearic acid salts, coconut oil acid or hydrogenated coconut oil acid; acyl lactylates in which the acyl radical contains from 8 to 20 carbon atoms. It is also possible to use alkyl D-galactoside uronic acids and salts thereof, polyoxyalkylenated (C₆-C₂₄)alkyl ether carboxylic acids, polyoxyalkylenated (C₆-C₂₄)alkylaryl ether carboxylic acids, and polyoxyalkylenated (C₆-C₂₄)alkylamido ether carboxylic acids and salts thereof, such as those containing from 2 to 50 alkylene oxide groups, for example, ethylene oxide groups, and mixtures thereof.

(ii) Nonionic surfactant(s):

Nonionic surfactants useful herein are known in the art. See, for example, “Handbook of Surfactants” by M. R. Porter, published by Blackie & Son (Glasgow and London), 1991, pp. 116-178). The nature of the nonionic surfactants is not a critical factor in the context of the present disclosure. Thus, may be chosen from the following non-limiting list: polyethoxylated or polypropoxylated alkylphenols and alpha-diols or alcohols, having a fatty chain containing, for example, from 8 to 18 carbon atoms, wherein the number of ethylene oxide or propylene oxide groups ranges from 2 to 50. The nonionic surfactants may also be chosen from polymers of ethylene oxide and of propylene oxide, condensates of ethylene oxide and of propylene oxide with fatty alcohols; polyethoxylated fatty amides having, for example, from 2 to 30 mol of ethylene oxide, polyglycerolated fatty amides comprising on average from 1 to 5, for example, from 1.5 to 4 glycerol groups; polyethoxylated fatty amines, for example, having from 2 to 30 mol of ethylene oxide; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, and amine oxides such as (C₁₀-C₁₄)alkylamine oxides or N-acylaminopropylmorpholine oxides.

(iii) Amphoteric or zwitterionic surfactant(s):

The amphoteric or zwitterionic surfactants may be chosen from aliphatic secondary or tertiary amine derivatives in which the aliphatic radical is a linear or branched chain having from 8 to 18 carbon atoms and having at least one water-solubilizing anionic group (for example carboxylate, sulfonate, sulfate, phosphate or phosphonate), (C₈-C₂₀)alkylbetaines, sulfobetaines, (C₈-C₂₀)alkylamido(C₁-C₆)-alkylbetaines, and (C₈-C₂₀)alkylamido(C₁-C₆)alkylsulfobetaines.

Among the amine derivatives that may be used, are those sold under the name Miranol®, described in U.S. Pat. Nos. 2,528,378 and 2,781,354 and classified in the CTFA dictionary, 3rd edition, 1982, under the names amphocarboxyglycinates and amphocarboxypropionates, having the respective structures:
R_{d}—CONHCH₂CH₂—N(Rₑ)(R_{f})(CH₂COO31⁻)
wherein R_{d} is chosen from an alkyl radical of an acid R_{d}—COOH present in hydrolyzed coconut oil, a heptyl, nonyl, and undecyl radical; Rₑ is a beta-hydroxyethyl group; and R_{f} is a carboxymethyl group; and
R_{g}—CONHCH₂CH₂—N(B)(C)
wherein B is —CH₂CH₂OX, C is —(CH₂)_{z}—Y, wherein z=1 or 2, X is chosen from a —CH₂CH₂—COOH group and a hydrogen atom; Y is chosen from —COOH and a —CH₂—CHOH—SO₃H radical, and R_{g} is an alkyl radical of an acid Rₕ-COOH present in coconut oil or in hydrolyzed linseed oil, a saturated radical or a radical comprising one or more unsaturations, such as a C₇ to C₁₇ radical, for example, a C₉, C₁₁, C₁₃ or C₁₇ alkyl radical or its iso form, and an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium caprylamphodipropionate, lauroamphodipropionic acid, and cocoamphodipropionic acid.

For example, the cocoamphodiacetate sold under the trade name Miranol® C2M concentrate by the company Rhodia Chimie may also be used.

(iv) Cationic Surfactants:

Cationic surfactants that may be used include salts of optionally polyoxyalkylenated primary, secondary, and tertiary fatty amines; quaternary ammonium salts such as tetraalkylammonium, alkylamidoalkyltrialkylammonium, trialkylbenzylammonium, trialkylhydroxyalkylammonium, and alkylpyridinium chlorides and bromides; imidazoline derivatives; and cationic amine oxides.

The amount of surfactants present in the composition may range from about 0.01% to about 40% by weight, such as from 0.5% to 30% by weight, relative to the total weight of the composition.

Medium

The cosmetically acceptable medium of the composition may be an aqueous medium comprising water and may further comprise at least one cosmetically acceptable organic solvent including, for example, C₁-C₄ alcohols such as ethyl alcohol, isopropyl alcohol, aromatic alcohols such as benzyl alcohol and phenylethyl alcohol, and glycols and glycol ethers such as, for example, ethylene glycol monomethyl, monoethyl and monobutyl ether, propylene glycol and ethers thereof such as, for example, propylene glycol monomethyl ether, butylene glycol, dipropylene glycol, and diethylene glycol alkyl ethers, for example diethylene glycol monoethyl ether and monobutyl ether, and polyols, for example glycerol. Polyethylene glycols, polypropylene glycol, and mixtures the compounds listed above may also be used as solvent.

The at least one solvent may be present in an amount ranging from 0.5% to 20% by weight, for example, from 2% to 10% by weight, relative to the total weight of the composition.

Additives

The cosmetic compositions may also comprise an effective amount of other agents, which are known in the field of dyeing of human keratin fibers, such as thickeners, antioxidants, fragrances, dispersants, conditioners such as cationic and amphoteric polymers, opacifiers, sequestering agents such as EDTA and etidronic acid, UV-screening agents, waxes, volatile or non-volatile, cyclic or linear or branched silicones, which may be organomodified (such as by amine groups) or unmodified, preserving agents, ceramides, pseudoceramides, plant, mineral or synthetic oils, vitamins or provitamins, for example panthenol, and nonionic, anionic, amphoteric and cationic associative polymers.

A person skilled in the art will take care to select the optional additional compound or compounds mentioned above such that the advantageous properties intrinsically associated with the dye composition according to the disclosure are not, or are not substantially, adversely affected by the envisaged addition or additions.

In another embodiment, ready-to-use compositions comprising a composition as described above and at least one oxidizing agent are provided.

The oxidizing agent may be chosen from, for example, hydrogen peroxide, urea peroxide, alkali metal bromates and ferricyanides, persalts such as perborates and persulfates, and enzymes such as peroxidases and two- and four-electron oxidoreductases.

In one embodiment, hydrogen peroxide is used.

The oxidizing agent may comprise an aqueous hydrogen peroxide solution with a titer ranging from 1 to 40 volumes, such as from 5 to 40 volumes.

The at least one oxidizing agent may be present in an amount ranging from 0.001% to 10% by weight relative to the weight of the ready-to-use composition

The pH of the compositions described above including the ready-to-use compositions may range from 4 to 12, such as from 6 to 11.

The pH may be adjusted to a desired value using acidifying or basifying agents that are well known in the art of dyeing keratin fibers.

The basifying agents include, but are not limited to, aqueous ammonia, alkali metal carbonates, alkanolamines such as monoethanolamine, diethanolamine and triethanolamine, and derivatives thereof, oxyethylenated and/or oxypropylenated hydroxyalkylamines and ethylenediamines, sodium hydroxide, potassium hydroxide, and compounds of the formula: 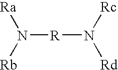
wherein R is chosen from a propylene residue optionally substituted with a hydroxyl group and a C₁-C₄ alkyl radical; Rₐ, R_{b}, R_{c}, and R_{d}, which may be identical or different, are each chosen from a hydrogen atom, C₁-C₄ alkyl radicals, and C₁-C₄ hydroxyalkyl radicals.

The acidifying agents include, but are not limited to, mineral or organic acids, such as hydrochloric acid, orthophosphoric acid, and carboxylic acids, for example tartaric acid, citric acid, lactic acid, and sulfonic acids.

As described above, the process according to the present disclosure comprises:
(a) applying a ready-to-use composition comprising at least one oxidation dye, at least one conductive polymer, and at least one oxidizing agent in a cosmetically acceptable medium to wet or dry fibers and leaving the composition on the fibers for a time sufficient to develop coloration;
(b) optionally rinsing the fibers,
(c) washing and rinsing the fibers, and
(d) drying the fibers or leaving the fibers to dry.

In one embodiment, the ready-to-use compositions are obtained by extemporaneous mixing, just before application to the fibers, of a dye composition comprising at least one oxidation dye and at least one conductive polymer with an oxidizing composition comprising at least one oxidizing agent in a suitable dyeing medium.

The oxidizing agent present in the oxidizing composition may be chosen from the agents listed above in the context of the definition of the ready-to-use compositions.

The medium of the oxidizing compositions may be water or a mixture of water and of at least one organic solvent. Suitable solvents are described in the description of the composition above.

The compositions may also comprise additives that are known in the field, for example surfactants, thickeners, antioxidants, fragrances, dispersants, conditioners, sequestering agents, preserving agents, etc.

According to another embodiment of the processes described herein, the ready-to-use compositions may be obtained by successively applying, in any order, at least one dye composition asdescribed above and at least one oxidizing composition as described above.

In accordance with one embodiment, the dye compositions described herein may be stored separately from the oxidizing composition. The dye compositions and the oxidizing composition are then mixed together at the time of use, the mixture is applied to keratin fibers, then the other steps of the process described above are performed.

A time that is sufficient to develop the coloration generally ranges from 1 to 60 minutes, such as from 5 to 45 minutes.

Moreover, step (a) of the process may be performed at a temperature ranging from 15 to 80° C., such as from 15 to 40° C.

Once step (a) is complete, the fibers may optionally be rinsed, washed with a shampoo, rinsed and then dried or left to dry at a temperature ranging from 20 to 120° C.

The invention is illustrated in greater detail by the examples described below. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

The examples that follow illustrate the disclosure but should not be construed as limiting the true scope of the disclosure, which is described in the specification and in the claims which follow.

### EXAMPLES

Synthesis of poly(thiophene-3-acetic acid)

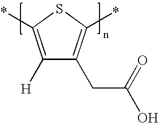

Procedure

Preparation of the Polymer: Poly(Ethyl Thiophene-3-Acetate)

25 ml of dry chloroform was introduced into a Schlenk tube under argon, the system was degassed, and 2.5 g of ethyl thiophene-3-acetate (14.7 mmol): 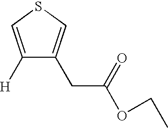
and 1 g of FeCl₃ (6.15 mmol) were then introduced. The mixture was stirred for 24 hours under argon at 50° C. The poly(ethyl thiophene-3-acetate) polymer was precipitated in heptane. The polymer was then dissolved in a tetrahydrofuran solution.

Infrared Characterization:

C═O band: 1719 cm⁻¹; CH₂, CH₃ bands=2979 cm⁻¹, 2934 cm⁻¹ and disappearance of the CH band at 3102 cm⁻¹ present in the monomer.

Hydrolysis of the Polymer: poly(ethyl thiophene-3-acetate) to form Poly(thiophene-3-acetic acid)

The polymer obtained above was hydrolyzed with an excess of 50 ml of an aqueous sodium hydroxide solution (2N) for 48 hours at 70° C., followed by acidification with concentrated HCl up to the point of precipitation of the poly(thiophene-3-acetic acid) product. The polymer was then filtered off and washed several times with distilled water to remove the traces of catalyst.

Infrared characterization of the polymer:

C═O band: 1740 cm⁻¹; COO band 1580 cm⁻¹; OH (broad band 3000-3500 cm⁻¹).

Neutralization of the poly(thiophene-3-acetic acid) polymer:

The poly(thiophene-3-acetic acid) polymer (2 g) was dissolved in tetrahydrofuran (30 g) and neutralized with a proportion of 1 mol of sodium hydroxide per mole of carboxylic acid. Water (30 g) was then added and the tetrahydrofuran was evaporated off. An aqueous 6% solution of poly(thiophene-3-acetic acid) in the form of a sodium salt was thus obtained.

Formulation comprising the Polymer obtained and dyeing process:

The dye formulation below was prepared:

| | |
|---|---|
| Poly(thiophene-3-acetic acid) | 5 g |
| para-Phenylenediamine | 0.216 g |
| 2-Methylaminophenol | 0.248 g |
| 20% aqueous ammonia | 9 g |
| Ethyl alcohol | 15 g |
| Water qs | 100 g |

20-volumes of aqueous hydrogen peroxide solution was added (1 dose of aqueous hydrogen peroxide solution per 1 dose of dye formula). After mixing together, the mixture was applied to locks of white hair. After a standing time of 20 minutes, light rinsing and drying (drying in the open air) was performed.

The locks were dyed beige and showed a particularly high sheen. When viewed at different angles, the locks exhibited different glints because pinks, yellows or greens were added to the beige shade.

The same test was performed, using the formula listed above but without poly(thiophene-3-acetic acid).

The locks were dyed beige, but had less sheen than the locks treated with the formulation comprising the conductive polymer. Furthermore, they did not show any change in glints as a function of the viewing angle.

1. A composition comprising, in a cosmetically acceptable medium:
(a) at least one oxidation dye, and
(b) at least one conductive polymer.

2. The composition according to claim 1, wherein the at least one conductive polymer comprises at least one repeating unit chosen from:
anilines of formula (I): 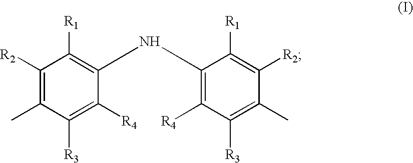
pyrroles of formulas (IIa) and (IIb): 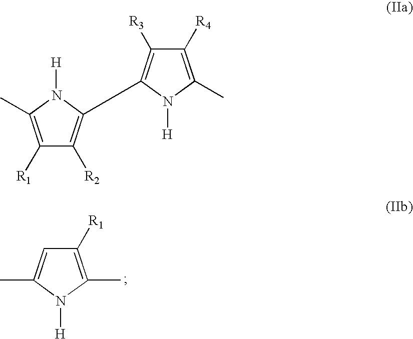
thiophenes and bisthiophenes of formulas (IIIa), (IIIb) and (IIIc): 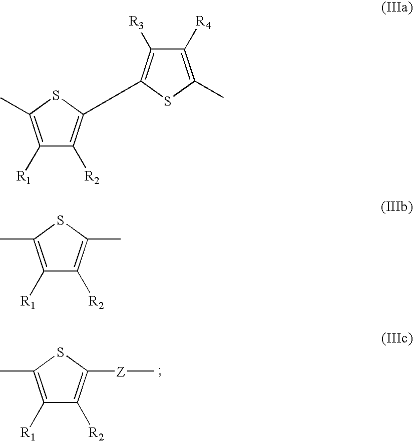
furans of formula (IV): 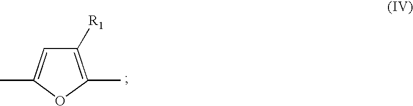
para-phenylene sulfides of structure (V): 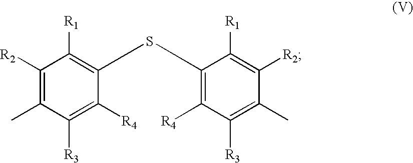
para-phenylenevinylenes of formula (VI): 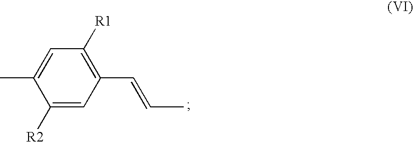
indoles of formula (VII): 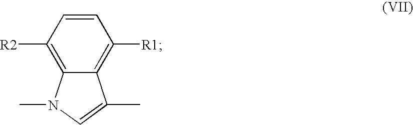
aromatic amides of formulas (VIIIa), (VIIIb), (VIIIc) and (VIIId): 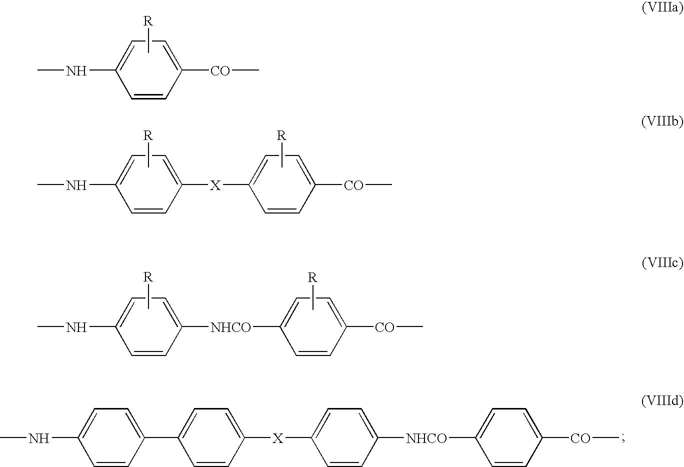
aromatic hydrazides of formulas (IXa), (IXb) and (IXc): 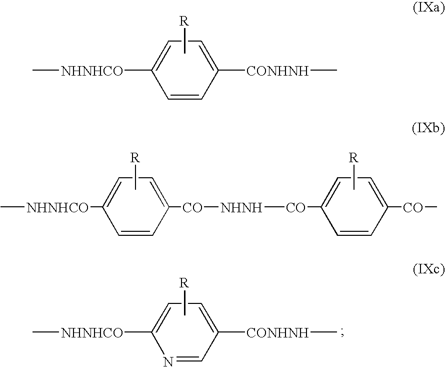
aromatic azomethines of formulas (Xa), (Xb) and (Xc): 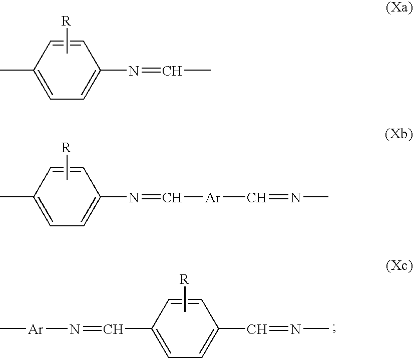
and
aromatic esters of formulas (XIa), (XIb) and (XIc): 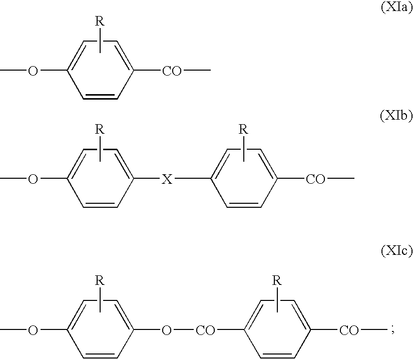
wherein the radicals R, R₁, R₂, R₃, and R₄, which may be identical or different, are each chosen from hydrogen, and from radicals —R′, —OR′, —COOR′, and —OCOR′, wherein R′ is chosen from linear and branched C₁-C₂₀ alkyl radicals, halogen atoms, nitro radicals, cyano radicals, cyanoalkyl radicals, and solubilizing groups optionally comprising a spacer group that bonds to the ring;
Ar is a radical comprising a monoaromatic or polyaromatic radical;
X is chosen from —O— and —S— atoms and from —NHCO—, —SO₂—, —N═N—, —C(CH₃)₂—, —CH₂—, —CH═CH—, and —CH═N— radicals; and
Z is chosen from —CH═CH— and —C≡C— radicals.

3. The composition according to claim 2, wherein the solubilizing groups are chosen from:
carboxylic acids —COOH,
carboxylate radicals —COO-M⁺wherein M⁺is chosen from alkali metals; alkaline-earth metals; organic amines, alkanolamines, and amino acids,
sulfonic acids —SO₃H,
sulfonate radicals —SO₃^{-M+,}
primary, secondary, and tertiary amine radicals,
quaternary ammonium radicals,
hydroxyl radicals, and
poly((C₂-C₃)alkylene oxide) radicals.

4. The composition according to claim 3, wherein the quaternary ammonium groups are —N(R′)₃^{+Z}⁻wherein Z is chosen from Br and Cl atoms and from (C₁-C₄)alkyl-OSO₃ radicals, and each R′, which may be identical or different, is chosen from linear and branched C₁ to C₂₀ alkyl radicals, or two of R′ form a heterocycle together with the nitrogen.

5. The composition according to claim 2, wherein the solubilizing groups are connected to the ring via a spacer group.

6. The composition according to claim 5, wherein the spacer group is chosen from —R″—, —OR″—, —OCOR″—, and —COOR″— radicals, wherein R″ is chosen from linear and branched C₁-C₂₀ alkyl radicals optionally comprising at least one heteroatom.

7. The composition according to claim 2, wherein the radicals R, R₁, R₂, R₃, and R₄, which may be identical or different, are each chosen from hydrogen, and R′, —OR′, —OCOR′, and —COOR′ radicals, wherein R′ is chosen from linear and branched C₁-C₆ alkyl radicals, and from the optionally neutralized solubilizing groups —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, and —[(CH₂)₂O]ₓCH₂CH₂OCH₃, wherein x is an average number ranging from 0 to 200.

8. The composition according to claim 2, wherein at least one of radicals R, R₁, R₂, R₃, and R₄ is a solubilizing group.

9. The composition according to claim 1, wherein the conductive polymer comprises at least one solubilizing group per repeating unit.

10. The composition according to claim 9, wherein the solubilizing groups are optionally connected to the ring via a spacer group and are chosen from optionally neutralized carboxylic acid groups; optionally neutralized sulfonic acid groups; tertiary amine radicals; quaternary ammonium radicals, and salts thereof.

11. The composition according to claim 10, wherein the solubilizing groups are chosen from quaternary ammonium radicals of the formula —N(R′)₃⁺Z⁻wherein Z is chosen from Br and Cl atoms, and from (C₁-C₄)alkyl-OSO₃ radicals, and each R′, which may be identical or different, is chosen from linear and branched C₁-C₂₀ alkyl radicals.

12. The composition according to claim 10, wherein the spacer group is chosen from C₁-C₂₀ alkyl radicals.

13. The composition according to claim 2, wherein the at least one conductive polymer is chosen from polymers comprising at least one repeating unit of formula (IIIa), (IIIb) or (IIIc), wherein at least one radical of R₁, R₂, R₃, and R₄ of formula (IIIa) or R₁ and R₂ of formulas (IIIb) or (IIIc) is chosen from optionally neutralized carboxylic acid solubilizing groups, optionally connected to the ring via a spacer group, and wherein the other radicals are hydrogen atoms.

14. The composition according to claim 13, wherein the spacer group is chosen from linear and branched C₁-C₂₀ alkyl radicals.

15. The composition according to claim 1, wherein the at least one conductive polymer is present in an amount of at least 0.001% by weight relative to the total weight of the composition.

16. The composition according to claim 1, wherein the at least one conductive polymer is present in an amount of up to 50% by weight relative to the total weight of the composition.

17. The composition according to claim 1, wherein the at least one conductive polymer is present in an amount ranging from 0.1% to 50% by weight relative to the total weight of the composition.

18. The composition according to claim 1, wherein the at least one oxidation dye comprises at least one oxidation base chosen from ortho-phenylenediamines, para-phenylenediamines, double bases, ortho-aminophenols, para-aminophenols, heterocyclic bases, and acid addition salts thereof.

19. The composition according to claim 18, wherein the at least one oxidation base is present in an amount ranging from 0.0005% to 12% by weight relative to the weight of the composition.

20. The composition according to claim 1, wherein the at least one oxidation dye comprises at least one coupler chosen from meta-aminophenols, meta-phenylenediamines, meta-diphenols, naphthols, heterocyclic couplers, and acid addition salts thereof.

21. The composition according to claim 20, wherein the at least one coupler is present in an amount ranging from 0.0001% to 10% by weight relative to the weight of the composition.

22. The composition according to claim 1, wherein the cosmetically acceptable medium is water or a mixture of water and at least one solvent.

23. The composition according to claim 22, wherein the at least one solvent is chosen from C₁-C₄ alcohols, aromatic alcohols, glycols, glycol ethers, and polyols.

24. The composition according to claim 1, further comprising at least one surfactant chosen from nonionic, anionic, cationic, amphoteric, and zwitterionic surfactants.

25. The composition according to claim 1, wherein the at least one conductive polymer has a conductivity ranging from 1×10⁻⁵ to 5×10⁵ siemens/cm.

26. The composition according to claim 25, wherein the at least one conductive polymer has a conductivity ranging from 1×10⁻³ to 1×10⁵ siemens/cm.

27. The composition according to claim 26, wherein the at least one conductive polymer has a conductivity ranging from 1×10⁻¹ to 1×10⁴ siemens/cm.

28. A ready-to-use composition comprising, in a cosmetically acceptable medium:
(a) at least one oxidation dye, and
(b) at least one conductive polymer,
and further comprising at least one oxidizing agent.

29. The ready-to-use composition according to claim 28, wherein the at least one oxidizing agent is chosen from hydrogen peroxide, urea peroxide, alkali metal bromates, alkali metal ferricyanides, persalts, and enzymes.

30. The composition according to claim 29, wherein the at least one oxidizing agent is a persalt chosen from perborates and persulfates.

31. A process for treating human keratin fibers comprising
(a) applying a ready-to-use composition comprising at least one oxidation dye, at least one conductive polymer, and at least one oxidizing agent in a cosmetically acceptable medium to the wet or dry fibers and leaving the composition on the fibers for a time sufficient to develop coloration;
(b) optionally rinsing the fibers,
(c) washing and rinsing the fibers, and
(d) drying the fibers or leaving the fibers to dry.

32. The process according to claim 31, wherein the human keratin fibers are hair.

33. The process according to claim 31, wherein the at least one conductive polymer comprises at least one repeating unit chosen from:
anilines of formula (I): 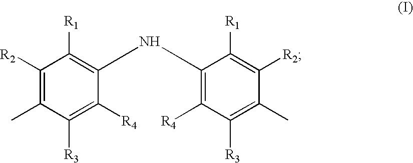
pyrroles of formulas (IIa) and (IIb): 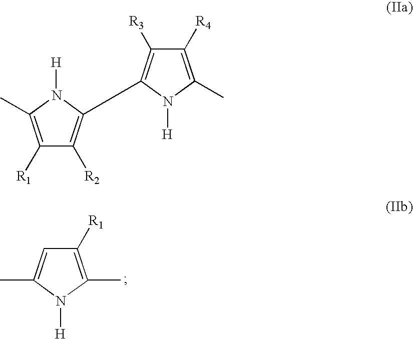
thiophenes and bisthiophenes of formulas (IIIa), (IIIb) and (IIIc): 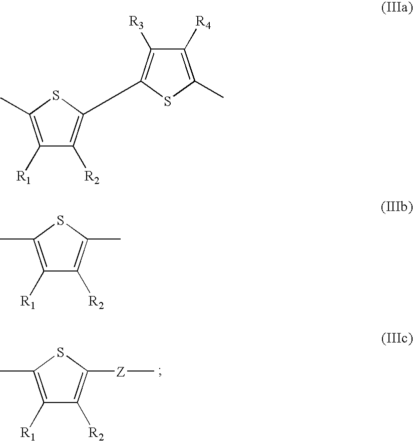
furans of formula (IV): 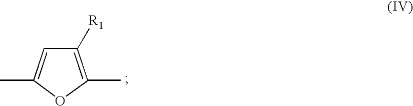
para-phenylene sulfides of structure (V): 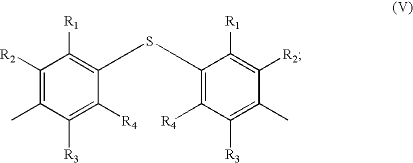
para-phenylenevinylenes of formula (VI): 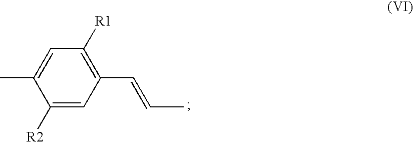
indoles of formula (VII): 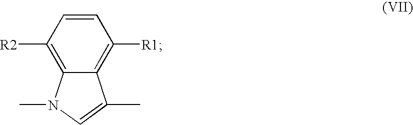
aromatic amides of formulas (VIIIa), (VIIIb), (VIIIc) and (VIIId): 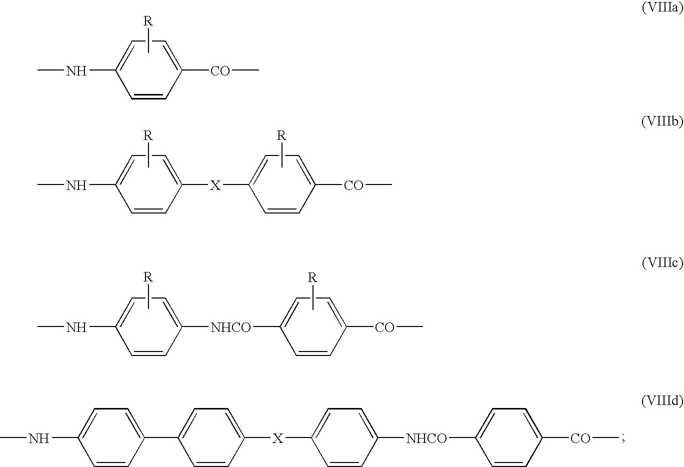
aromatic hydrazides of formulas (IXa), (IXb) and (IXc): 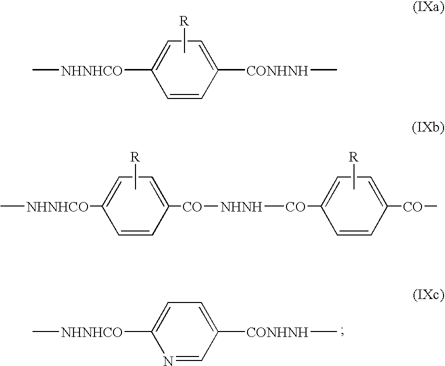
aromatic azomethines of formulas (Xa), (Xb) and (Xc): 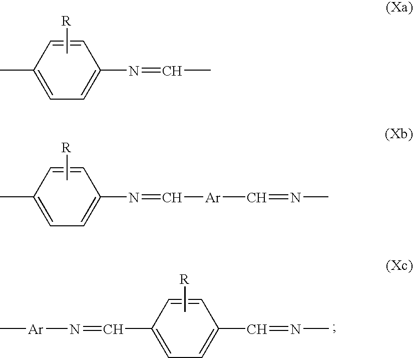
and aromatic esters of formulas (XIa), (XIb) and (XIc): 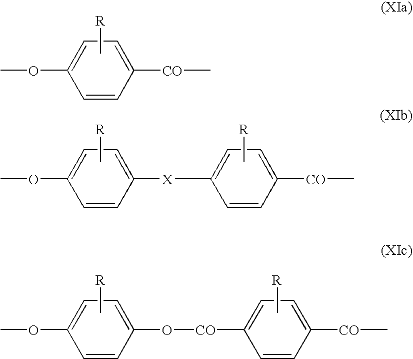
wherein the radicals R, R₁, R₂, R₃, and R₄, which may be identical or different, are each chosen from hydrogen, and from radicals —R′, —OR′, —COOR′, and —OCOR′, wherein R′ is chosen from linear and branched C₁-C₂₀ alkyl radicals, halogen atoms, nitro radicals, cyano radicals, cyanoalkyl radicals, and solubilizing groups optionally comprising a spacer group that bonds to the ring;
Ar is a radical comprising a monoaromatic or polyaromatic radical;
X is chosen from —O— and —S−, atoms and from —NHCO—, —SO₂—, —N═N—, —C(CH₃)₂—, —CH₂—, —CH═CH—, and —CH═N— radicals; and
Z is chosen from —CH═CH— and —C≡C— radicals.

34. The composition according to claim 33, wherein the solubilizing groups are chosen from:
carboxylic acids —COOH,
carboxylate radicals —COO-M⁺wherein M is chosen from alkali metals; alkaline-earth metals; organic amines, alkanolamines, and amino acids,
sulfonic acids —SO₃H,
sulfonate radicals —SO₃⁻M⁺,
primary, secondary, and tertiary amine radicals,
quaternary ammonium radicals,
hydroxyl radicals, and
poly((C₂-C₃)alkylene oxide) radicals.

35. The composition according to claim 34, wherein the quaternary ammonium groups are —N(R′)₃⁺Z⁻wherein Z is chosen from Br and Cl atoms and from (C₁-C₄)alkyl-OSO₃ radicals, and each R′, which may be identical or different, is chosen from linear and branched C₁ to C₂₀ alkyl radicals, or two of R′ form a heterocycle together with the nitrogen.

36. The process according to claim 31, wherein the ready-to-use composition is obtained by extemporaneous mixing, before application to the fibers, of a composition comprising at least one oxidation dye and at least one conductive polymer in a cosmetically acceptable medium with an oxidizing composition comprising at least one oxidizing agent in a suitable dyeing medium.

37. The process according to claim 31, wherein the ready-to-use composition is obtained by successively applying, in any order, a composition comprising at least one oxidation dye and at least one conductive polymer in a cosmetically acceptable medium and an oxidizing composition comprising at least one oxidizing agent in a suitable dyeing medium.

38. A method for imparting at least one optical effect on optical fibers comprising applying a composition comprising, in a cosmetically acceptable medium, at least one conductive polymer and at least one oxidation dye, wherein the at least one conductive polymer and the at least one oxidation dye are present in an amount sufficient to impart to the fibers an optical effect.

39. The method of claim 38, wherein the optical effect is a sheen effect.

40. A multicompartment kit comprising:
at least one first compartment comprising a dye composition comprising, in a cosmetically acceptable medium:
at least one oxidation dye, and
at least one conductive polymer, and
at least one second compartment comprising at least one oxidizing composition comprising at least one oxidizing agent.
